(19) United States
(12) Patent Application Publication
(10) Pub. No.: US 2014/0363500 A1
(43) Pub. Date: Dec. 11, 2014

(54) USE OF AN ANTIBODY AND A PARTICULATE IMMUNOMODULATOR

(71) Applicants: EPITARGET AS, Oslo (NO); UNIVERSITÉ CLAUDE BERNARD LYON I, Villeurbanne (FR); HOSPICES CIVILS DE LYON, Lyon (FR)

(72) Inventors: Charles DUMONTET, Lyon (FR); Esben A. NILSSEN, Oslo (NO)

(21) Appl. No.: 14/470,434

(22) Filed: Aug. 27, 2014

Related U.S. Application Data
(63) Continuation of application No. PCT/NO2013/050038, filed on Feb. 27, 2013.

(30) Foreign Application Priority Data
Feb. 27, 2012 (NO) 20120212

(51) Int. Cl. A61K 39/395; A61K 45/06; A61K 38/19; A61K 9/127; A61K 47/28

(52) U.S. Cl. CPC A61K 39/3955; A61K 9/127; A61K 47/28; A61K 38/193; A61K 45/06
USPC 424/450; 424/172.1; 424/489; 424/85.1; 424/85.2; 424/152.1; 424/145.1

(57) ABSTRACT

The current invention is directed to particulate or vesicular immunomodulators, like e.g. cytokines, for use in combination therapy with antibodies for treatments of a range of conditions and diseases, in particular cancer, as well as methods, compositions, and kits thereof.

### CROSS REFERENCE TO RELATED APPLICATIONS

This application is a Continuation of PCT International Application No. PCT/NO2013/050038 filed on Feb. 27, 2013 and claims priority under 35 U.S.C. 119(a) to Patent Application No. 20120212 filed in Norway on Feb. 27, 2012. This application also claims benefit under 35 U.S.C. 119(e) to U.S. Provisional Application No. 61/610,674 filed on Mar. 14, 2012. All of the above applications are hereby expressly incorporated by reference into the present application.

### FIELD OF THE INVENTION

The present invention is related to use of particulate immunomodulators, like e.g. cytokines, in combination therapy with antibodies for treating a range of conditions and disease states, in particular cancer, as well as methods, kits, and compositions thereof.

### BACKGROUND OF THE INVENTION

White blood cells are involved in a variety of host defence mechanisms. Innate immune cells constitute a primary defence barrier against infectious agents while adaptive immunity provides a highly focused and powerful response. Cellular components of these responses involve a variety of leucocytes, including polymorphonuclear cells, monocytes and macrophages, and lymphocytes. These cells are also susceptible to participate in antitumor responses, although the development of tumours in a host is usually associated with a suppression of these potential cellular effectors. Suppression may be either non-specific, with a reduction of migration or phagocytic properties, or specific with deletion or inhibition of tumour-specific cells. A possibility for therapeutic intervention thus consists in the specific stimulation of cellular subtypes. The most classical is vaccination, which induces a highly targeted antigen-specific response. It is becoming increasingly clear that the global modulation of a leucocyte subpopulation could be of interest in the treatment of certain diseases such as cancer. Potential examples of this approach which are not yet applied in the clinic include the suppression of T regulatory cells which facilitate tumourigenesis or the stimulation of polymorphonuclear cells which are involved in so-called antibody dependent cellular cytotoxicity (ADCC) of therapeutic monoclonal antibodies (mAbs).

mAbs like e.g. rituximab, trastuzumab, and cetuximab are currently in routine clinical use to treat a range of diseases and medical conditions including cancer, cardiovascular disease, transplant rejection, psoriasis, etc. The exact mode of action of mAbs is still unclear, but in general they are thought to work by inhibiting their target molecule, complement activation, and/or by inducing ADCC. In the latter case, NK cells, monocytes, macrophages, and neutrophils expressing the Fcγ receptor (FcγR), in particular FcγRIII, bind to the Fc region of the mAb inducing a response ultimately killing the target cell. Although mAbs have had a great impact on medical therapy since their introduction into the clinics in the late nineties, the therapeutic response of mAb treatment is still suboptimal and, in the case of cancer, reduced or completely eliminated by development of tumour resistance in the patient.

Growth factors, like e.g. the cytokine granulocyte-colony stimulating factor (G-CSF), are currently used in medical therapy to decrease the impacts of chemotherapy-induced neutropenia. From in vitro studies it has, however, long been known that cytokines like e.g. granulocyte-colony stimulating factor (G-CSF), granulocyte macrophage colony stimulating factor (GM-CSF), and interleukin-2 (IL-2) also potentiate ADCC. See e.g. Honsik et al. (1986); Munn and Cheung (1987); Kushner and Cheung (1989); Ottonello et al. (1999); Stockmeyer et al. (2001); van der Kolk et al. (2002). Studies in animal xenograft models of non-Hodgkin lymphoma (NHL) have corroborated these in vitro data (Hernandez-Ilizaliturri et al. 2003; Hernandez-Ilizaliturri et al. 2005; Lopes de Menezes et al. 2007), however, a clear clinical picture has not emerged: van der Kolk et al. (2003) could not provide support for G-CSF improving efficacy of rituximab in a study of relapsed low grade lymphoma patients, while Niitsu et al. (2005) reported that the treatment effect of combining G-CSF with the rituximab-EPOCT (rituximab with etoposide, vincristine, pirarubicine, cyclophosphamide, and prednisone) regimen did not appear to diverge from earlier studies with rituximab and cytostatics. By contrast, a retrospective study by Gruber et al. (2011) strongly indicated prolonged progression free survival in chronic lymphocytic leukaemia patient after addition of G-CSF to a fludarabine, cyclophosphamide, and rituximab regimen. Recently, Cartron et al. (2008) reported a phase II study testing GM-CSF in combination with rituximab in patients with relapsed follicular lymphoma with encouraging results. Here, the combination appeared to increase the complete response rate compared to rituximab alone. Several clinical studies are underway to further investigate a potential cytokine-mAb synergy in cancer patients.

Fast clearance and nonspecific biodistribution in vivo limits clinical use of cytokines. For example, G-CSF (filgrastim) has an elimination half-life of only 3.5 hrs. in humans. There are several approaches to improve the pharmacokinetics and modify the biodistribution of cytokines, including e.g. conjugation to polyethylene glycol (PEG; pegylation) or albumin, as well as encapsulation into particles or vesicles. Pegylation of filgrastim has been particularly successful, increasing the elimination half-life from 15 to 80 hrs. Another interesting approach has been liposomal encapsulation of cytokines. The focus areas of the liposome protagonists have typically been reduced biodistribution to irrelevant tissues, plasma clearance, and toxicity. Use of particulate targeting to leucocyte subpopulations would be of great value since it would allow both the use of considerably smaller doses and a reduced exposure of non-target tissues. Particulate distribution of therapeutic agents has been validated in a number of instances, for example in the case of liposomal anticancer agents such as doxorubicin or potentially nephrotoxic antimycotic agents such as amphotericin B.

Debs and coworkers (Debs et al. 1990) report liposomal Tumour Necrosis Factor α (TNF-α). The liposomes may comprise lipids phosphatidylserine (PS), phosphatidylglycerol (PG), or phosphatidylcholine (PC). All liposomes comprise cholesterol and a heterogeneous size distribution with a mean diameter of 2.03 μm is reported. No therapeutic advantage of liposomal TNF-α compared to free TNF-α was recorded.

Anderson and co-workers (Anderson et al. 1990) describe multilamellar vesicles consisting of DMPC, DMPG, and interleukine-2 (IL-2) for treatment of sarcoma pulmonary metastases.

Furthermore, Anderson and co-workers (Anderson et al. 1994) disclose liposomal cytokines comprising dimyristoylphosphatidylcholine (DMPC) and one of the cytokines IL-1, IL-2, IL-6, GM-CSF, or IFN-γ.

Meyer and coworkers (Meyer et al. 1994) report liposomal G-CSF comprising the phospholipids dimyristoylphosphatidylglycerol (DMPG), DMPC, and cholesterol.

Rourke and coworkers (Rourke et al. 1996) disclose liposomal formulations of G-CSF, pSt, IL-2, IL-4, and GM-CSF comprising one of the phospholipids DMPG, dipalmitoylphosphatidylglycerol (DPPG), dioleoylphosphatidylglycerol (DOPG), DMPS, DOPS, DMPC, DPPC, DOPC.

Kedar and co-workers disclose a range of different liposomal formulations of IL-2, GM-CSF, TNF-α through four publications (Kedar et al. 1994a; Kedar et al. 1994b; Kedar et al. 1997; Kedar et al. 2000). In the last paper of the series it is concluded that large multilamellar vesicles consisting of DMPC and without steric stabilisation, that is, without polyethylene glycol (PEG), have a stronger immunomodulatory activity than sterically stabilised small egg PC based liposomes.

None of the publications supra mention or suggest the added therapeutic benefit of using PE based liposomes or high PEG concentrations in liposomal cytokines, nor the strong synergistic activity between liposomal cytokines, in particular liposomal G-CSF, and mAb.

One of current applicants has earlier disclosed liposomes comprising unsaturated phospholipids comprising anti-cancer peptides or proteins, like filgrastim, pegfilgrastim, or sargramostim (WO 2009/075582, WO 2010/143969, WO 2010/143970). The therapeutic use of such liposomal cytokines without acoustic triggering is neither mentioned nor suggested, further, the current combinatorial use of liposomal cytokines and mAbs is not disclosed.

Although the first identified publications on liposomal cytokines are more than 20 years old, no liposomal cytokines have so far reached the market. The current inventors have found that certain particulate or vesicular formulations of cytokines have a dramatic therapeutic effect when coadministered with therapeutic monoclonal antibodies, in some cases producing curative results in animal tumour models with very low concentrations of liposomal cytokine. It is an important aspect of the current invention that the antibodies as not conjugated or associated with the liposomal cytokine, rather, the liposomal cytokine is a standalone drug provided to patients referred to e.g. mAb therapy. Thus, the present invention may be used to improve cancer therapy of a range of current and future therapeutic mAbs.

### Definitions

DOPE herein means 1,2-Dioleoyl-sn-Glycero-3-Phosphoethanolamine

DSPC means 1,2-distearoyl-sn-glycero-3 phosphocholine or, in short, distearoylphosphatidylcholine.

DSPE means 1,2-distearoyl-sn-glycero-3-phosphoethanolamine or distearoylphosphatidylethanolamine.

DSPE-PEGXXXX means 1,2-distearoyl-sn-glycero-3-phosphoethanolamine-N-[methoxy(polyethylene glycol)-XXXX, wherein XXXX signifies the molecular weight of the polyethylene glycol moiety, e.g. DSPE-PEG2000 or DSPE-PEG5000.

IS herein mean Inverted Structure.

n-alcohol means any alcohol with n carbon atoms.

PC herein means phosphatidylcholine with any composition of acyl chain.

PE means phosphatidylethanolamine with any composition of acyl chain length.

PEG means polyethylene glycol or a derivate thereof.

PEGXXXX means polyethylene glycol or a derivate thereof, wherein XXXX signifies the molecular weight of the polyethylene glycol moiety.

POPE herein means 1-palmitoyl-2-oleoyl-sn-glycero-3-phosphoethanolamine.

SOPE herein means 1-stearoyl-2-oleoyl-sn-glycero-3-phosphoethanolamine.

‘Immunomodulator’ herein means a substance which influences either the absolute number or functions of leucocytes or a certain subpopulation of leucocytes.

### General Provisions

The phospholipid, cholesterol, PEG-lipid concentrations mentioned herein are nominal values unless stated otherwise.

In the current disclosure singular form means singular or plural. Hence, ‘a particle’ may mean one or several particles. Furthermore, all ranges mentioned herein includes the endpoints, e.g. the range ‘from 14 to 18’ includes 14 and 18.

### DETAILED DESCRIPTION OF THE INVENTION

The current inventors have found that the therapeutic combination or co-administration of certain particulate or vesicular formulations of immunomodulators and antibodies dramatically improves the therapeutic response in diseased animals, in particular in animals with cancer.

One aspect of the current invention relates to a particulate or vesicular material comprising an immunomodulator for use in combination therapy with an antibody in treatment of a condition or a disease.

The invention further relates to a combination of an antibody and a particulate or vesicular material comprising an immunomodulator for use in treatment of a condition or a disease, wherein the antibody and the material is not directly and stably associated or forming one molecular entity.

The invention also relates to use of a combination of an antibody and a particulate or vesicular material comprising of an immunomodulator for manufacturing a medicament for treating a condition or a disease, wherein the antibody and the material is not directly associated or forming one molecular entity.

Another aspect of the invention is use of a particulate or vesicular material comprising an immunomodulator for manufacturing a medicament for treating a condition or a disease, wherein said material is combined or co-administered with an antibody.

A further aspect of the invention is a combination comprising an antibody and a particulate or vesicular material comprising an immunomodulator.

One aspect of the invention is directed to a composition including an antibody and a particulate or vesicular material including an immunomodulator.

Yet another aspect of the invention is a pharmaceutical composition comprising an antibody and a particulate material comprising an immunomodulator, wherein the antibody is not conjugated or directly associated with said particulate material.

A further aspect of the invention is a kit including an antibody and a particulate or vesicular material including an immunomodulator.

Another aspect of the invention is directed to a method of treating a disease or a condition including administering to a subject in need thereof a composition including an antibody and a particulate material including an immunomodulator.

Another aspect of the invention is directed to a method of treating cancer including administering to a subject in need thereof a pharmaceutical composition including a therapeutic monoclonal antibody and a liposome encapsulating a cytokine, wherein the liposome includes a phoshatidylethanolamine (PE) or phosphatidylserine (PS), cholesterol, and polyethylene glycol (PEG) or a derivative thereof.

A further aspect of the invention is directed to a pharmaceutical composition including a therapeutic monoclonal antibody and a liposome encapsulating granulocyte-colony stimulating factor (G-CSF), wherein the liposome includes at least 52 mol % of 1,2-Dioleoyl-sn-Glycero-3-Phosphoethanolamine (DOPE).

Yet another aspect of the current invention is a method of treatment comprising administering an antibody and a particulate material comprising an immunomodulator to a patient in need thereof.

The invention also relates to a method of producing the combination, composition, or kit.

It is important to note that the antibody is not conjugated through e.g. a covalent bond or in any other way directly and stably associated with the particulate or vesicular immunomodulator. Thus, in a clinical setting the antibody and the particulate or vesicular immunomodulator may be administered separately, at same or different points in time, or in the form of a ready made composition, at the discretion of the medical practitioner. It is, however, essential that the antibody is not forming a stable molecular entity (e.g. a liposome comprising both cytokines and mAb conjugated or stably associated with the liposomal membrane) with the particular or vesicular immunomodulator of the current invention

The particulate or vesicular material or formulation may be arranged in any form of dispersion of a given internal structure. Examples of preferred structures are hexagonal structures (e.g. Hexosome®), cubic structures (e.g. Cubosomes®), emulsion, microemulsions, micelles, liquid crystalline particles, or liposomes. According to a preferred embodiment, the particulate material is a membrane structure, more preferably a liposome. A liposome normally consists of a lipid bilayer with an aqueous interior. Preparation of liposomes is well known within the art and a number of methods may be used to prepare the current material.

Said particulate or vesicular material or formulation may further comprise any lipid. Preferably, the lipid is an amphiphilic lipid such as a sphingolipid and/or a phospholipid. In a preferred embodiment the amphiphilic lipids are phospholipids of any type or source.

The phospholipid may be saturated or unsaturated, or a combination thereof, however, the phospholipids are preferably unsaturated. Typically, the selected phospholipids will have an acyl chain length at least 12 carbon atoms, more often at least 14 carbon atoms, and more often at least 16 carbon atoms, and even more often at least 18 carbon atoms. Preferably the acyl chain length is within the range 14 to 24 carbon atoms, more preferably 14 to 22 carbon atoms, even more preferably within 16 to 22 carbon atoms, even more preferably within 16 to 18. Acyl chain of different lengths may be mixed in the material of the invention, including asymmetric phospholipids, or all acyl chains may have similar or identical length. In preferred embodiments of the current invention the acyl chain length of the phospholipid is either 18 carbon atoms or a mixture of phospholipids of acyl chain length 16 and 18 carbon atoms.

Furthermore, the polar head of the phospholipid may be of any type, e.g. phosphatidylethanolamine (PE), phosphatidylcholine (PC), phosphatidic acid (PA), phosphatidyl serine (PS), phosphatidylglycerol (PG), phosphatidylinositol. Also, the material or formulation may comprise mixtures of phospholipids with different polar heads. However, the lipids or phospholipids may not be derivatised to polyethylene glycol, like in e.g. DSPE-PEG, unless explicitly stated. At last one acyl chain may be unsaturated, however, it is preferred that both acyl chains are unsaturated. Lysolipids, like lysoPE, may also be included in the material of the invention. The phospholipid is preferably PE, PC, PG, and/or PS, more preferably PE, PC, and/or PS, even more preferably unsaturated PE, PC, and/or PS, even more preferably unsaturated PE and/or PS, and most preferably unsaturated PE. Preferred PEs are listed in Table 1 and 2, while preferred PCs are listed in Table 3 and 4. In embodiments of the current invention the phospholipid is 1,2-Dioleoyl-sn-Glycero-3-Phosphoethanolamine (DOPE), 1,2-Dioleoyl-sn-Glycero-3-Phosphoserine (DOPS), 1,2-Dioleoyl-sn-Glycero-3-Phosphocholine (DOPC), or hydrogenated soy PC. In a preferred embodiment of the current invention the phospholipid is DOPE.

The phospholipid concentration in the material or formulation of the current invention may be of any suitable level. Typically, the PC concentration will be within the range 50 to 80 mol %, preferably within the range 50 to 60 mol %. In one embodiment of the invention the PC concentrations, more specifically, the HSPC concentration, is about 57 mol %. However, if the PC is unsaturated, higher concentrations of PC are preferred. The PE or PS concentration should preferably be within the range 25 to 98 mol %, more preferably within the range 32 to 98 mol %, even more preferably 32 to 75 mol %, even more preferably 40 to 75 mol %, even more preferably 50 to 75 mol %, even more preferably 52 to 75 mol %. Medium to higher PE or PS concentrations are preferred, e.g. at least 40 mol %, more preferably at least 50 mol %, more preferably at least 60 mol % PE. Accordingly, in preferred embodiments of the current invention the PE concentration is about 54, 58, or 62 mol %, while the PS concentration is 62 mol %.

TABLE 1
Symmetric PE

| Carbon number | Product |
|---|---|
| 16:0 | Dipalmitoyl PE (DPPE) |
| 16:0[(CH3)4] | Diphytanoyl PE |
| 16:1 | Dipalmitoleoyl PE |
| 17:0 | Diheptadecanoyl PE |
| 18:0 | Distearoyl (DSPE) |
| 18:1(delta 9-cis) | Dioleoyl (DOPE) |
| 18:1(delta 9-trans) | Dielaidoyl |
| 18:2 | Dilinoeoyl |
| 18:3 | Dilinolenoyl |
| 20:4 | Diarachidonoyl |
| 22:6 | Docosa-hexaenoyl |

TABLE 2
Asymmetric PE

| Carbon number | 1-Acyl | 2-Acyl |
|---|---|---|
| 16:0-18:1 | Palmitoyl | Oleoyl (POPE) |
| 16:0-18:2 | Palmitoyl | Linoleoyl |
| 16:0-20:4 | Palmitoyl | Arachidonoyl |
| 16:0-22:6 | Palmitoyl | Docosahexaenoyl |
| 18:0-18:1 | Stearoyl | Oleoyl (SOPE) |
| 18:0-18:2 | Stearoyl | Linoleoyl |
| 18:0-20:4 | Stearoyl | Arachidonoyl |
| 18:0-22:6 | Stearoyl | Docosahexaenoyl |

TABLE 3
Symmetric PC

| Carbon number | Trivial | IUPAC |
|---|---|---|
| 16:0 | Dipalmitoyl PC (DPPC) | Dihexadecanoyl PC |
| 16:1 | Dipalmitoleoyl PC | 9-cis-hexadecenoyl PC |
| 18:0 | Distearoyl PC | Dioctadecanoyl pC |
| 18:1 | Petroselinoyl PC | 6-cis-octadecenoic PC |
| 18:1 | Oleoyl PC (DOPC) | 9-cis-octadecenoic PC |
| 18:1 | Elaidoyl PC | 9-trans-octadecenoic PC |
| 18:2 | Linoleoyl PC | 9-cis-12-cis- |
| | | octadecadienoic PC |
| 18:3 | Linolenoyl PC | 9-cis-12-cis-15- |
| | | cisoctadecatrienoic PC |
| 20:1 | Eicosenoyl PC | 11-cis-eicosenoic PC |
| 20:4 | Arachidonoyl PC | 5,8,11,14(all -cis) |
| | | eicosatetraenoic PC |
| 22:1 | Erucoyl PC | 13-cis-docosenoic |
| 22:6 | DHA PC | 4,7,10,13,16,19 (all -cis) |
| | | docosahexaenoic PC |
| 24:1 | Nervonoyl PC | 15-cis-tetracosenoic PC |

TABLE 4
Asymmetric PC

| Carbon Number | 1-Acyl | 2-Acyl |
|---|---|---|
| 18:0-18:1 | Stearoyl | Oleoyl |
| 18:0-18:2 | Stearoyl | Linoleoyl |
| 18:0-20:4 | Stearoyl | Arachidonoyl |
| 18:0-22:6 | Stearoyl | Docosahexaenoyl |

Components or stabilising agents for improving blood circulation time and/or further modulate efficacy, improve shelf life, etc, may be included in the material, like e.g. poly(oxazoline), polyvinyl alcohol, poly (glycerol), poly-N-vinylpyrrolidone, poly[N-(2-hydroxypropyl)methacrylamide], poly(amino acid)s, dextran, polyethylene glycol (PEG), or polymers. More specifically, the material or formulation may comprise e.g. polyvinyl alcohols, polyethylene glycols (PEG), dextrans, or other polymers or derivates thereof conjugated or associated to a molecule, e.g. a lipophilic molecule, to obtain anchoring to the current particulate material. PEG or a derivate thereof, at any suitable concentration, is preferred. An example of a PEG derivative would be 1,2-distearoyl-glycol)-2000 (DSPE-PEG200). However, PEG concentrations are preferably at least 2 mol %, more preferably at least 5 mol %, even more preferably at least 8 mol %, within the range 3 to 20 mol %, even more preferably within the range 4 to 20 mol %, and even more preferably within the range 8 to 20 mol %. In embodiments of the current invention the PEG concentration is 5, 8, 12, or 16 mol %. 8, 12, or 16 mol % are preferred, and the range 8 to 16 mol % is consequently particularly preferred. The PEG moiety may be of any molecular weight or type, however, it is preferred that the molecular weight is within the range 100 to 5000 Da, more preferably within 350-5000 Da, even more preferably 2000-5000 Da. In preferred embodiments the molecular weight is 2000 Da or 5000 Da. The PEG moiety may be associated with any molecule allowing it to form part of the particulate or vesicular material. Preferably, the PEG moiety is conjugated to a sphingolipid (e.g. ceramide), a glycerol based lipid (e.g. phospholipid), or a sterol (e.g. cholesterol), more preferably to a ceramide and/or PE, and even more preferably to PE, like DMPE, DPPE, or DSPE. The lipid-grafted PEG is preferably 1,2-distearoyl-sn-glycero-3-phosphoethanolamine-N-[methoxy(polyethylene glycol)-2000] (DSPE-PEG 2000) and/or 1,2-distearoyl-sn-glycero-3-phosphoethanolamine-N-[methoxy(polyethylene glycol)-5000] (DSPE-PEG 5000). In preferred embodiments of the current invention the lipid-grafted PEG is DSPE-PEG 2000 or DSPE-PEG5000.

The particulate material may also comprise a sterol. The particulate material may comprise any suitable sterol concentration, preferably cholesterol, depending on the specific particle properties. In general, 50 mol % sterol is considered the upper concentration limit in liposome membranes. The current particulate material preferably comprises up to 20 mol % cholesterol, more preferably up to 30 mol %, and even more preferably up to 40 mol % cholesterol, more preferably cholesterol concentrations within the range 1-40 mol %, more preferably 10-40 mol %, even more preferably 20-40 mol %, and most preferably within the range 30 to 40 mol %. In preferred embodiments of the current invention the particulate material comprises 20, 30, 38 or 40 mol % cholesterol. Accordingly, the cholesterol concentration is preferably within the range constituted by any of the mentioned embodiment concentrations.

In preferred embodiments the material has the composition DOPE:DSPE-PEG2000:Chol (mol %) 62:8:30, 58:12.30, 54:16:30, or 72:8:20.

The particulate material of the invention may be of any suitable size. However, the material should preferably have an average diameter (as measured by dynamic light scattering) within the range 50 to 2000 nm, more preferably with in the range 50 to 1200 nm, even more preferably within the average size range 80 to 800 nm, even more preferably within the range 80 to 400 nm, even more preferably within the range 80 to 200 nm. In embodiments of the current invention the average size is typically within the ranges 80 to 510 nm. The size distribution may be narrow or wide. Typically, all particulate material should be within the range 50 to 1500 nm.

Any immunomodulator may be associated with the current formulations. The association may be through strong chemical bonds, like e.g. covalent bonds, or weak bond, or hydrophilic or lipophilic interactions. For example, the cytokine may be conjugated to DSPE-PEG via a maleimide moiety. It is preferred that the association between immunomodulator and the current material is of non-covalent or weak nature, e.g. hydrophilic or lipophilic interactions. An ‘immunomodulator’ is defined as a substance which influences either the absolute number or functions of leucocytes or a certain subpopulation of leucocytes. The immunomodulator may be any immunomodulatory molecule, preferably a peptide or a protein, more preferably a cytokine. Examples of cytokines are colony-stimulating factor (CSF), interferon (IFN), interleukin (IL), stem cell factor (SCF), tumour growth factors (TGF), and tumour necrosis factor (TNF). Preferably, the cytokine is a CSF, IL, IFN, or any combination thereof; more preferably, the cytokines are CSF and/or IL; and most preferred the cytokine is a CSF. The CSF may be chosen from one or any combination of the CSFs ancestim, garnocestim, pegacaristim, leridistim, milodistim, filgrastim, lenograstim, nartograstim, pegfilgrastim, pegnartograstim, ecogramostim, molgramostim, regramostim, sargramostim, cilmostim, lanimostim, mirimostim, daniplestim, muplestim, or derivates thereof.

The CSF may be a G-CSF, M-CSF, and/or GM-CSF, although G-CSF and GM-CSF are preferred. Any type of G-CSF or GM-CSF may be used alone or in combination, like e.g. filgrastim, lenograstim, nartograstim, pegfilgrastim, pegnartograstim, ecogramostim, molgramostim, regramostim, sargramostim, and/or derivates thereof, although, the G-CSFs filgrastim or lenograstim are preferred. In preferred embodiments of the current invention the G-CSF is filgrastim or lenograstim. The interleukin may be of any sort and source. At present at least 35 major interleukins have been identified named from IL-1 to IL-35. Preferably the IL is IL-2 and/or IL-4, most preferably IL-2 like aldesleukin or a derivate thereof. An example of a derivative of aldesleukin would be PEG-aldesleukin.

The concentration of cytokine in the material of the invention may vary according to the therapeutic goals. For example, free G-CSF is generally dosed at 5-10 μg/kg/day for an expected duration of 14 days. In a mouse (20 g) this corresponds to a dose of approximately 60-120 μg/kg/day, or a total weekly dose of approx. 840 μg/kg. The current inventors have shown that a weekly dose of cytokine (including both liposomal and extraliposomal cytokine) as low as 37.5 μg/kg when formulated as herein described is superior to a weekly dose of 900 μg/kg of free G-CSF. Accordingly, the lipid/cytokine concentration ratio should be at least 10,000. By way of example, if the lipid concentration of a liposome (including phospholipids, PEG phospholipids, and cholesterol) is 30 mg/ml, then the cytokine concentration should be at least 3 μg/ml. In one embodiment of the current invention the nominal lipid/cytokine ratio is 4,000.

The current inventors have found that a co-administration of the particulate or vesicular material described herein with an antibody may produce a dramatic improvement in therapeutic efficacy. The material discussed herein may be co-administered with the antibody in the same dosage form, pharmaceutical formulation, or composition, however, it is preferred that the particulate or vesicular formulation of immunomodulator and the antibody is administered separately. Hence, it is preferred that the two entities are provided in two separate dosage forms. The dosage forms may, however, be supplied as a kit consisting of a particulate formulating of immunomodulator and an antibody.

Without being bound to current scientific theory, the inventors believe that the fc region is necessary for the current combination to work efficaciously. Hence, the antibody may be of any type and source, however, it is preferably an IgG antibody, even more preferably an IgG1 or IgG2, more specifically an IgG2a, antibody; even more preferably an IgG1 antibody; or an IgG derivate thereof. The antibody is preferably monoclonal and it will typically be a so-called therapeutic antibody. The anti body should preferably target one, two, three, four, or more of the following targets: CD20, CD52, CD3, CD4, CD5, CD8, CD19, CD22, CD38, CD138, HER2, ErbB2, CD11, CD30, CD33, CD52, to CD25, vascular endothelial growth factor (VEGF), epidermal growth factor receptor (EGFR), Insulin-like Growth Factor 1 (IGF1) receptor or CTLA-4. Antibodies targeting CD20 may be type I or type II, although antibodies with improved ADCC are generally preferred. In embodiments of the current invention the antibodies target CD20, HER2, or EGFR.

The antibody, one, two, three, four, or more, may be selected from the following group: abciximab, adalimumab, alemtuzumab, atlizumab, basiliximab, belimumab, bevacizumab, brentuximab vedotin, canakinumab, cetuximab, certolizumab pegol, cixutumumab, daclizumab, denosumab, eculizumab, efalizumab, farletuzumab, gemtuzumab, golimumab, ibritumomab tiuxetan, infliximab, ipilimumab (MDX-101), muromonab-CD3, natalizumab, necitumunab, obinutuzumab (GA-101), ocaratuzumab (AME-133v), ocrelizumab, ofatumumab, omalizumab, palivizumab, panitumumab, pertuzumab, PRO131921, ranibizumab, rituximab, SBI-087, tocilizumab, TRU-015, tositumomab, trastuzumab, zalutumumab, or veltuzumab. The antibody is preferably alemtuzumab, ocaratuzumab (AME-133v), ocrelizumab, bevacizumab, cetuximab, cixutumab, denosumab, gemtuzumab, ibritumomab tiuxetan, ipilimumab, obinutuzumab (GA-101), ocaratuzumab (AME-133v), ocrelizumab, ofatumumab, panitumumab, pertuzumab, PRO131921, rituximab, SBI-087, trastuzumab, TRU-015, tocilizumab, tositumomab, tocilizumab or veltuzumab, or any combination thereof; even more preferably, cetuximab, cixutumab, ocrelizumab, obinutuzumab (GA-101), pertuzumab, PRO131921, rituximab, trastuzumab, ofatumumab, tocilizumab, or any combination thereof; yet even more preferably obinutuzumab (GA-101), rituximab, cetuximab, and/or trastuzumab; yet even more preferably rituximab, obinutuzumab (GA-101), or trastuzumab. In preferred embodiments of the current invention the antibody is rituximab or trastuzumab.

The particulate material of the invention may further comprise an additional drug or a functional molecule of any sort. The drug may be any drug suitable for the purpose. However, anti-bacterial drugs, anti-inflammatory drugs, immunosuppressive drugs, anti cancer drugs, or any combination thereof are preferred. As the current technology is particularly adapted for treating cancer, anti cancer drugs are preferred. Anti cancer drugs includes any chemotherapeutic, cytostatic or radiotherapeutic drug. It may be of special interest to load the current particulate material with deoxyribonucleic acid (DNA) or ribonucleic acid (RNA), in particular small interfering RNA (siRNA).

The general groups of cytostatics are alkylating agents (L01A), anti-metabolites (L01B), plant alkaloids and terpenoids (L01C), vinca alkaloids (L01CA), podophyllotoxin (L01CB), taxanes (L01CD), topoisomerase inhibitors (L01CB and L01XX), antitumour antibiotics (L01D), platinum compounds, recombinant enzymes, hormonal therapy. Examples of cytostatics are gemcitabine, daunorubicin, cisplatin, docetaxel, 5-fluorouracil, vincristine, methotrexate, cyclophosphamide, L-asparaginase and doxorubicin.

Accordingly, the drug may include alkylating agents, antimetabolites, anti-mitotic agents, epipodophyllotoxins, antibiotics, hormones and hormone antagonists, enzymes, platinum coordination complexes, anthracenediones, substituted ureas, methylhydrazine derivatives, imidazotetrazine derivatives, cytoprotective agents, DNA topoisomerase inhibitors, biological response modifiers, retinoids and arsenic derivatives, therapeutic antibodies, differentiating agents, immunomodulatory agents, and angiogenesis inhibitors.

The drug may also be alpha emitters like e.g. radium-223 (223Ra) and/or thorium-227 (227Th) or beta emitters like yttrium-90. Other alpha emitting isotopes currently used in preclinical and clinical research include astatine-211 (211At), bismuth-213 (213Bi), and actinium-225 (225Ac).

Moreover, the drug may further comprise anti-cancer peptides, like telomerase or fragments of telomerase, like hTERT; or proteins, like monoclonal or polyclonal antibodies, scFv, tetrabodies, Vaccibodies, Troybodies, etc. Also, the material of the invention may comprise collagenases or other enzymes targeting the microenvironmental stroma, tumor endothelium, or surface antigens of tumor cells, particular proteins or molecules improving the uptake and distribution of particulate material in target tissues.

More specifically, therapeutic agents that may be included in the particulate material include abarelix, alemtuzumab, alitretinoin, allopurinol, altretamine, amifostine, anastrozole, arsenic trioxide, asparaginase, BCG live, bexarotene, bleomycin, bortezomib, busulfan, calusterone, camptothecin, capecitabine, carboplatin, carmustine, celecoxib, chlorambucil, cinacalcet, cisplatin, cladribine, cyclophosphamide, cytarabine, dacarbazine, dactinomycin, darbepoetin alfa, daunorubicin, denileukin diftitox, dexrazoxane, docetaxel, doxorubicin, dromostanolone, Elliott's B solution, epirubicin, epoetin alfa, estramustine, etoposide, exemestane, floxuridine, fludarabine, fluorouracil, fulvestrant, gemcitabine, gemtuzumab ozogamicin, gefitinib, goserelin, hydroxyurea, ibritumomab tiuxetan, idarubicin, ifosfamide, imatinib, interferon alfa-2a, interferon alfa-2b, irinotecan, letrozole, leucovorin, levamisole, lomustine, meclorethamine, megestrol, melphalan, mercaptopurine, mesna, methotrexate, methoxsalen, methylprednisolone, mitomycin C, mitotane, mitoxantrone, nandrolone, nofetumomab, oblimersen, oprelvekin, oxaliplatin, paclitaxel, pamidronate, pegademase, pegaspargase, pemetrexed, pentostatin, pipobroman, plicamycin, polifeprosan, porfimer, procarbazine, quinacrine, rasburicase, streptozocin, talc, tamoxifen, tarceva, temozolomide, teniposide, testolactone, thioguanine, thiotepa, topotecan, toremifene, tretinoin, uracil mustard, valrubicin, vinblastine, vincristine, vinorelbine, zoledronate, and elaidic acid ester of cytarabine.

Drugs may be modified by addition of a lipid. Such lipophilic drug should preferably comprise a long hydrocarbon chain and/or a hydrophobic ring structure. The hydrocarbon chain of the lipophilic drug is preferably at least 18 carbon atoms long. Preferably the hydrocabon chain is an elaidic acid. Most preferably, the lipophilic drug is an elaidic acid ester of gemcitabine, cytarabine, betamethason, prednisolon, acyclovir, ganciclovir, or ribavirin.

Furthermore, the particle of the invention may also comprise an imaging contrast agent, like e.g. an MR, X-ray, or optical imaging contrast agent, to render tracking and monitoring possible or a gamma-emitter for nuclear imaging. Examples of MR and X-ray contrast agents, as well as fluorescent and bioluminescent probes may be found in the literature.

The vesicle or particle of the invention does preferably not comprise any bubbles or undissolved gases, like e.g. found in microbubbles.

Another aspect of the current invention is a therapeutic or medical method for treating a disease or a condition comprising the steps of administering the particulate material and the antibody both as described herein to a subject in need thereof. The therapeutic method may be used to treat a range of diseases and conditions where elimination or reduction of specific cells in the diseased body is needed.

As used herein a subject includes a bird, reptile, amphibian or mammal, such as a human, or a pet, for example, a cat or a dog, a farm animal, such as a pig, sheep, goat, horse, or cow.

Activation of, or triggered release from, vesicles, microbubbles, liposome, or any other particulate material or entity by means of acoustic energy, ultrasound, or heat is preferably not part of the current invention.

The condition or disease is preferably allergy, asthma, cancer, cardiovascular disease, autoimmune disorders, transplant rejection, infectious diseases, inflammatory diseases, degenerative diseases, haematological diseases, myalgic encephalopathy, chronic fatigue syndrome, post viral fatigue syndrome, rheumatoid arthritis; more preferably, cancer, cardiovascular disease, rheumatoid arthritis, or autoimmune disorders. In a preferred embodiment of the current invention the disease or condition is cancer. Any type of cancer may be treated depending on the specificity of the antibody, as exemplified by the use of rituximab, trastuzumab, and cetuximab in the current embodiments. Rituximab, trastuzumab, and cetuximab targets antigens CD20, HER2, and EGFR, respectively, and may be used to treat all cancer forms expressing, preferably overexpressing, said antigens. In current clinical practice rituximab is used to treat e.g. lymphomas, in particular non-Hodgkin lymphoma or follicular non-Hodgkin lymphoma, chronic lymphatic leukaemia, rheumatoid arthritis; trastuzumab's main medical indications are adjuvant treatment of HER2 overexpressing breast cancer, HER2 positive metastatic breast cancer, and metastatic gastric cancer; while cetuximab is indicated for head and neck cancer, more specifically locally or regionally advanced squamous cell carcinoma of the head and neck, as well as EGFR-expressing colorectal cancer. Accordingly, the conditions or diseases of the current application is preferably lymphoma, chronic lymphatic leukaemia, rheumatoid arthritis, breast cancer, gastric cancer, head and neck cancer, colorectal cancer, more preferably, follicular non-Hodgkin lymphoma or HER2 positive breast cancer.

### BRIEF DESCRIPTION OF DRAWINGS

FIG. 1. Liposomal G-CSF and rituximab treatment of lymphoma SCID mice carrying a RL xenograft, a follicular non-Hodgkin's lymphoma model, treated with liposomal G-CSF and the monoclonal antibody rituximab. Control groups are untreated control, rituximab only, and liposomal G-CSF only. The combination of liposomal G-CSF and rituximab has a dramatic effect on the growth of the tumour.

FIG. 2. Efficacy of liposomal G-CSF in combination with trastuzumab in Her2 positive tumour (A549)

SCID mice carrying a A549 xenograft, a Her2 positive lung carcinoma model, treated with liposomal G-CSF and the monoclonal antibody trastuzumab. Control groups are untreated control, trastuzumab only, and liposomal G-CSF only. The combination of liposomal G-CSF and trastuzumab completely inhibits tumour growth.

FIG. 3. Efficacy of liposomal G-CSF in combination with rituximab in rituximab resistant RL (non-Hodgkin's lymphoma) xenograft model.

SCID mice carrying a rituximab resistant RL xenograft, a follicular non-Hodgkin's lymphoma model, treated with liposomal G-CSF and the monoclonal antibody rituximab. Control groups are untreated control, rituximab only, and liposomal G-CSF only. The combination of liposomal G-CSF and rituximab appears to overcome the resistance of the model and delay tumour growth significantly.

### EXAMPLES

### Example 1

### Preparation of DOPE Based Liposomes Comprising Cytokine

DOPE and DSPE-PEG 2000 were purchased from Genzyme Pharmaceuticals (Liestal, Switzerland). Cholesterol, HEPES, HSPC, TRITON-X100 (10% solution), sodium azide and sucrose were obtained from Sigma Aldrich. G-CSF was purchased from Chugai Pharmaceuticals (Granocyte™ lenograstim) or Teva Pharmaceuticals (Tevagrastim™; filgrastim).

G-CSF carrying liposomes (liposomal G-CSF) of different membrane composition were prepared using the thin film hydration method (Lasic 1993). Briefly, liposome components were dissolved in a chloroform/methanol mixture (9/1 v/v) at 60° C. and rotary evaporated to dryness under vacuum for 6 h. The resulting dried lipid films were hydrated with G-CSF (for concentrations, see batch table) dissolved in phosphate buffered saline (PBS; pH 7.4) solution for 2-6 h followed by three freeze-thaw cycles in a dry ice/acetone/methanol mixture and water, respectively. The liposomes at a lipid concentration of 30 mg/ml were extruded (Lipex extruder, Biomembrane Inc., Vancouver B.C., Canada) through Nucleopore polycarbonate filters with pore sizes of 800 nm (Nucleopore, West Chester, Pa., USA). The extruder, including filters, should be flushed with buffer comprising 2 ml/ml serum (e.g. BSA) before use. The lipid hydration, liposome extrusion and thawing process were performed above the gel-to-liquid-crystalline phase transition temperature of the phospholipids. For production of small sized liposomes the liposomes were downsized by stepwise extrusion through Nucleopore polycarbonate filters with pore sizes of 800, 400, 200, 100 and 80 nm.

Extraliposomal G-CSF may be removed by e.g. dialysis, diafiltration, or size exclusion chromatography, although this is not generally necessary. Dialysis was performed by placing disposable dialysers (MW cut off 100 000 D) containing the liposome dispersion, in a large volume of PBS solution (pH 7.4). The setup was protected from light and the dialysis ended when the trace of G-CSF in the dialysis was negligible. The liposome dispersion was then, until further use, stored in the fridge protected from light.

TABLE 1
Liposomal Cytokine Batches

| Batch no. | Composition mol % | Conc. μg/ml (cytokine) | Lipid conc mg/ml | Average size nm (pdi) |
|---|---|---|---|---|
| 1 (218) | 62:8:30 (DOPE:DSPE- | 7.5 (lenograstim) | 30 | 246 (0.37) |
| | PEG2000:Chol) | | | |
| 2 (219) | 62:8:30 (DOPE:DSPE- | 0 | 30 | 504 (0.48) |
| | PEG2000:Chol) | | | |
| 3 (235) | 62:8:30 (DOPE:DSPE- | 7.5 (lenograstim) | 30 | 273 (0.34) |
| | PEG2000:Chol) | | | |
| 4 (236) | 62:8:30 (DOPE:DSPE- | 0 | 30 | 297 (0.4) |
| | PEG2000:Chol) | | | |
| 5 (252) | 62:8:30 mol % (DOPE:DSPE- | 7.5 (lenograstim) | 30 | 277 (0.37) |
| | PEG2000:Chol) | | | |
| 6 (253) | 62:8:30 mol % (DOPE:DSPE- | 7.5 (lenograstim) | 30 | 90 (0.06) |
| | PEG2000:Chol) | | | |
| 7 (254) | 57:6:38 mol % (HSPC:DSPE- | 7.5 (lenograstim) | 30 | 354 (0.18) |
| | PEG2000:Chol) | | | |
| 8 (255) | 57:6:38 mol % (HSPC:DSPE- | 7.5 (lenograstim) | 30 | 89 (0.04) |
| | PEG2000:Chol) | | | |
| 9 (256) | 62:8:30 mol % (DOPE:DSPE- | 7.5 (filgrastim) | 30 | 368 (0.29) |
| | PEG2000:Chol) | | | |
| 10 (262) | 62:8:30 mol % (DOPE:DSPE- | 45.3 (filgrastim) | 30 | 347 (0.50) |
| | PEG2000:Chol) | | | |
| 11 (263) | 62:8:30 mol % (DOPE:DSPE- | 60 (filgrastim) | 30 | 180 (0.28) |
| | PEG2000:Chol) | | | |
| 12 (283) | 52:8:40 mol % (DOPE:DSPE- | 10 (lenograstim) | 30 | 371 (0.30) |
| | PEG 2000:Chol) | | | |
| 13 (264) | DOPE:DSPE-PEG | Filgrastim | 30 | 111 (0.1) |
| | 2000:Chol 62:8:30 | 80 ug/ml | | |
| 14 (270) | DOPE:DSPE-PEG | Filgrastim | | 207 (0.36) |
| | 2000:Chol 62:8:30 mol % | 40 ug/ml | | |
| 15 (271) | DOPE:DSPE-PEG | Filgrastim | | 122 (0.06) |
| | 2000:Chol 62:8:30 | 40 ug/ml | | |
| 16 (272) | DOPE:DSPC:DSPE-PEG | Lenograstim | | 369 (0.31) |
| | 2000:Chol 62:10:8:20 | 10 ug/ml | | |
| 17 (273) | DOPE:DSPC:DSPE-PEG | Lenograstim | | 373 (0.51) |
| | 2000:Chol 62:10:8:20 | 40 ug/ml | | |
| 18 (274) | DOPE:DSPC:DSPE-PEG | Lenograstim | | 91.7 (0.07) |
| | 2000:Chol 62:10:8:20 | 40 ug/ml | | |
| 19 (283) | DOPE:DSPE-PEG | Lenograstim | | 371 (0.30) |
| | 2000:Chol 52:8:40 | 10 ug/ml | | |
| 20 (311) | DOPC:DSPE-PEG | Lenograstim | | 124 (0.084) |
| | 2000:Chol 62:8:30 | 10 ug/ml | | |
| 21 (312) | DOPS:DSPE-PEG | Lenograstim | | 131 (0.056) |
| | 2000:Chol 62:8:30 | 10 ug/ml | | |
| 22 (313) | DOPE:LysoPE:DSPE-PEG | Lenograstim | | 136 (0.084) |
| | 2000:Chol 52:10:8:30 | 10 ug/ml | | |
| 23 (314) | DOPE:DSPE-PEG | Lenograstim ≈ | | 192 (0.253) |
| | 2000:DSPE-PEG-MAL:Chol | 30 ug/ml | | |
| | 62:8:0.03:30 | | | |
| 24 (315) | DOPE:DSPE-PEG | Lenograstim ≈ | | 200 (0.258) |
| | 2000:DSPE-PEG-MAL:Chol | 30 ug/ml | | |
| | 62:8:0.03:30 | | | |
| 25 (316) | DSPE-PEG | Lenograstim ≈ | | |
| | 2000:DSPE-PEG-MAL | 10 ug/ml | | |
| | 62:8:0.03:30 | | | |
| 26 (317) | DOPE:DSPC:DSPE-PEG | Lenograstim ≈ | | 127 (0.126) |
| | 2000:DSPE-PEG-MAL:Chol | 30 ug/ml | | |
| | 62:10:8:0.1:20 | | | |
| 27 (328) | DOPE:DSPE-PEG | Lenograstim | | 128 (0.172) |
| | 2000:Chol 54:16:30 | 10 ug/ml | | 108 (0.167) |
| 28 (330) | DOPE:DSPE-PEG | Lenograstim | | 233 (0.259) |
| | 5000:Chol 62:8:30 | 10 ug/ml | | 206 (0.253) |
| 29 (331) | DOPE:DSPE-PEG | Lenograstim | | 603 (0.405) |
| | 2000:Chol 58:12:30 | 10 ug/ml | | |

### Example 2

### Characterisation of Liposomal G-CSF

Liposomes were characterised with respect to key physicochemical properties like particle size and osmolality by use of well-established methodology.

The average particle size (intensity weighted) and size distribution were determined by photon correlation spectroscopy (PCS) at a scattering angle of 173° C. and 25 deg C. (Nanosizer, Malvern Instruments, Malvern, UK). The width of the size distribution is defined by the polydispersity index. Prior to sample measurements the instruments was tested by running a latex standard (60 nm). For the PCS measurements, 5 μL of liposome dispersion (lipid conc. 30 mg/ml) was diluted with 2 mL sterile filtered isosmotic PBS solution (pH 7.4). Duplicates were analysed.

Osmolality was determined on non-diluted liposome dispersions by freezing point depression analysis (Fiske 210 Osmometer, Advanced Instruments, MA, US). Prior to sample measurements, a reference sample with an osmolality of 290 mosmol/kg was measured; if not within specifications, a two-step calibration was performed. Duplicates of liposome samples were analysed.

### Example 3

### RL Cell Line and Culture

The RL cell line, derived from a human transformed FL sample, was purchased and used as a model of Non-Hodgkin's Lymphoma (NHL) expressing CD20 antigen. Cells were maintained in culture medium consisting of RPMI-1640 (Life Technologies), 10% of fetal calf serum (Integro), 100 units/mL of penicillin and 100 mg/mL of streptomycin (Life Technologies). All cells were cultured at 37° C. in a 5% CO2 atmosphere.

### Example 4

### In Vivo Studies

Six-week-old female CB17 severe combined immune-deficient mice (SCID) mice purchased from Charles River laboratories (l'Arbresle) were bred under pathogen-free conditions at the animal facility of our institute. Animals were treated in accordance with the European Union guidelines and French laws for the laboratory animal care and use. The animals were kept in conventional housing. Access to food and water was provided ad libitum. This study was approved by the local animal ethical committee. Rituximab (MabThera™, Roche) and trastuzumab (Herceptin™, Roche) were purchased from the Pharmacy at Oslo University Hospital, Norway.

For lymphoma xenograft experiments, 1×10⁶ RL cells were injected subcutaneously on day 1. Mice were randomized into study groups when tumour volume was approximately 200 mm³.

A549 Her2 positive xenografts were surgically implanted and animals were randomized into study groups at a tumour size of approximately 20 mm3.

Animals were weighed and the tumour size was measured twice a week with an electronic calliper. The tumour volume (TV) was estimated from two dimensional tumour measurements by the formula: tumour volume (mm3) 1/4 length (mm) width 2/2. Median tumor growth inhibition (% TGI) was calculated according to the NCI formula: 1 ([TVtreated (day 34 20) 100/TVcontrol (day 34 20) 100]).

### Example 5

### Therapy Study Results: Combination of Rituximab and Liposomal G-CSF in a Non-Hodgkin's Lymphoma Model

Twelve SCID mice carrying the RL tumour (see above) was randomized into four study groups: (1) untreated control, (2) rituximab only, (3) liposomal G-CSF (Lipo-G; Batch #1), and (4) rituximab+Lipo-G. Rituximab was injected intraperitoneally (IP) at a dose of 100 mg/kg (200 μl injection volume), while Lipo-G was administered intravenously (IV) in the tail vein at a G-CSF dose of 37.5 μg/kg (100 μl injection volume). The injections were performed once a week for a duration of four weeks. All treatments were given on the same day (i.e. liposomal G-CSF and rituximab were given on the same day as weekly doses).

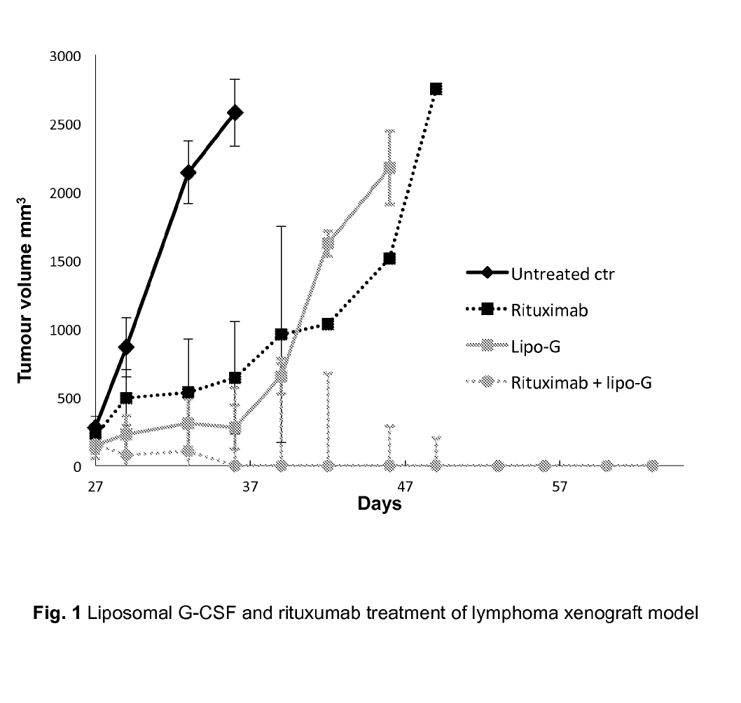

Tumour measurements were performed by caliper measurement of the diameter of the tumour (see above). See FIG. 1 for data presentation. Data represents the median with standard error mean. Untreated groups showed rapid tumour growth and were sacrificed after 29 days due to size of tumour. Administration of rituximab alone or Lipo-G (liposomal G-CSF) led to a reduction in rate of tumour growth, but all animals were sacrificed after 47 days. Combination treatment of rituximab and liposomal G-CSF results in complete remission of tumours. Mice were followed up for a total of 105 days after tumour initiation (Data points beyond 63 days not shown); no palpable tumour was detected throughout the follow-up period.

### Example 6

### Therapy Study Results: Comnbination of Trastuzumab and Liposomal G-CSF in a HER2 Positive Cell Line

The study was performed using a Her2 positive cell line, AK549, in SCID mice. A549 cells were surgically implanted into the right flank of 24 animals. Tumours were allowed to establish for 11 days prior to randomization into four study groups: (1) untreated control, (2) liposomal G-CSF (Lipo-G; Batch #3) (3) trastuzumab only (Trastuzumab), and (3) trastuzumab and liposomal G-CSF (Trastuzumab+Lipo-G)

Trastuzumab was administered at a concentration of 5 mg/ml. Animals received an IP injection of 100 μl providing an approximate dose of 1.5 mg per mouse (75 mg/kg given an animal weight of 20 g). Animals received 2 administrations per week for 4 weeks. Liposomal G-CSF (Batch #3) was administered in a PBS solution with a total G-CSF concentration (unencapsulated and encapsulated) of 7.5 μg/mL. Animals received an intravenous administration of 100 μl liposomal G-CSF constituting an approximate total dose of 37.5 μg/kg G-CSF (including both liposomal and nonliposomal G-CSF). Animals received liposomal G-CSF at the same time as trastuzumab administration; therefore mice received 2 administrations per week for 4 weeks. Dosing commenced at Day 11 after tumour implantation. Tumours were measured weekly until day 34 post-implantation. Each treatment group consisted of 6 animals.

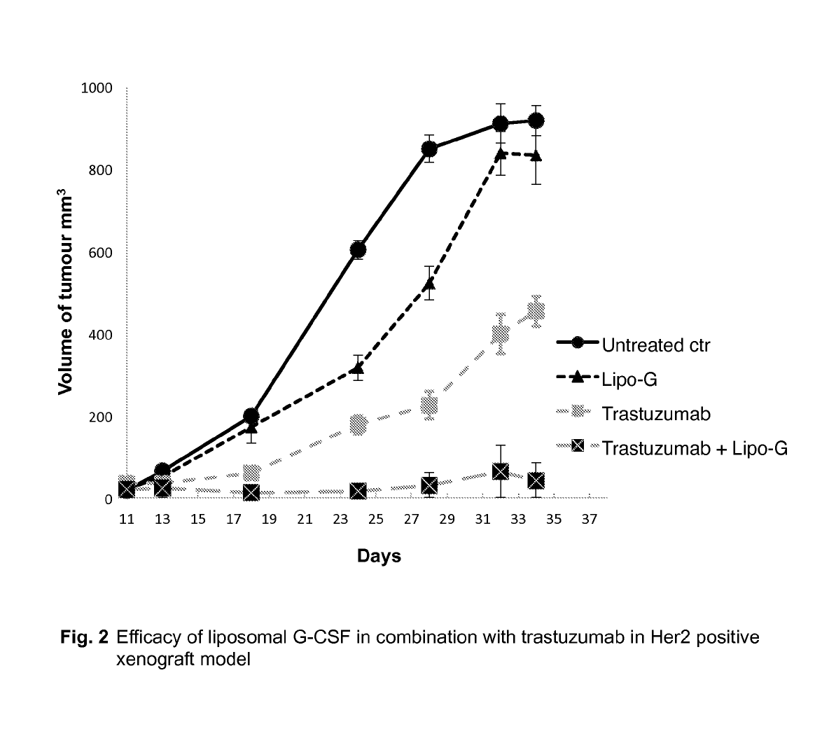

In untreated control animals tumours grew rapidly from a median size of 17 mm³ at D11 to 917 mm³ at D34. The tumours treated with the combination therapy showed almost no growth in the reporting period; the median tumour volume at D11 was 20 mm³ and grew to 42 mm³ at D34. Tumours treated with liposomal G-CSF (Lipo-G) alone showed tumour growth similar to untreated control. Trastuzumab alone resulted in reduced tumour growth, but combination therapy was greatly superior in its ability to reduce tumour growth. See FIG. 2 for data presentation.

### Example 7

### Therapy Study in Rituximab Resistant Tumour Model

The aim of this study was to investigate the effect of combinations treatment with both liposomal G-CSF (Formulation #12) and rituximab in a rituximab resistant model.

To establish the resistant model, lymphoma cell line RL is serially passaged in mice exposed to rituximab. Tumour cells obtained from mice having received rituximab were reinjected the same day to a new group of mice that were then treated with rituximab. After 5 passages, RL are resistant and the tumour growth with rituximab treatment is comparable with normal RL without treatment. At day one of the experiment, 7×10⁶ RL cells are reimplanted in complete RPMI (10% Foetal calf serum, 1% penicillin/streptomycin) in the right flank of SCID mice. Tumours are grown to an approximate size ranging from 100-200 mm³ before initiation of treatment.

Sixteen SCID mice carrying the resistant RL tumour (see above) was randomized into four study groups: (1) untreated control, (2) rituximab only, (3) liposomal G-CSF (Lipo-G; Formulation #12), and (4) rituximab+Lipo-G. Rituximab was injected intraperitoneally (IP) at a dose of approximately 30 mg/kg (doses were not corrected for individual animal weight, average animal weights were 20 g), while Lipo-G was administered intravenously (IV) in the tail vein at a G-CSF dose of 50 μg/kg (100 μl injection volume). The injections were performed once a week for a duration of three weeks (injection days 17, 25, and 31). All treatments were given on the same day (i.e. LipoG and rituximab were given on the same day as weekly doses).

Tumour measurements were performed by caliper measurement of the diameter of the tumour as describe above.

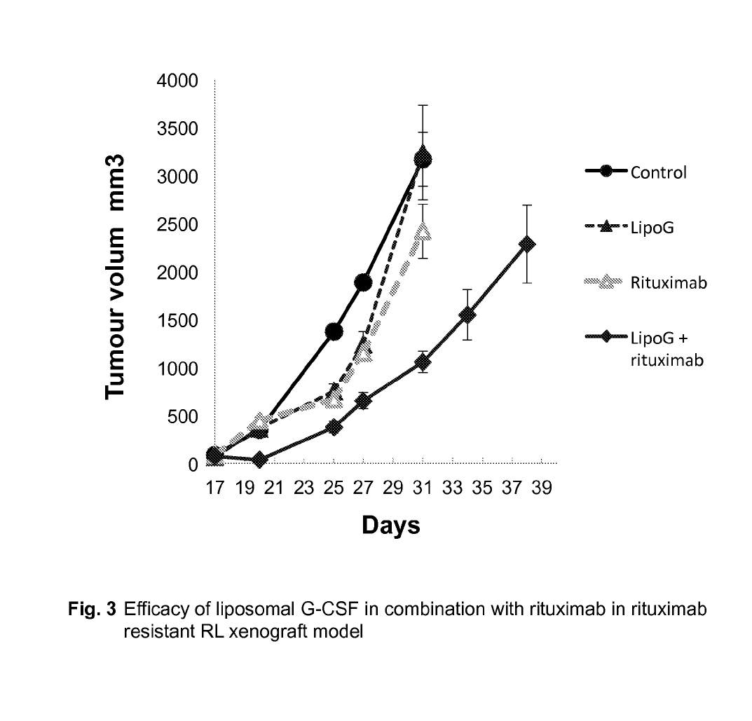

The data (FIG. 3) show that the group receiving both rituximab and LipoG injections had slower tumour growth compared to the groups receiving only rituximab or only LipoG. We conclude that combination treatment with rituximab and LipoG has efficacy in this rituximab resistant model.

### REFERENCES

Anderson, P. M., D. C. Hanson, D. E. Hasz, M. R. Halet, B. R. Blazar and A. C. Ochoa (1994). “Cytokines in Liposomes: Preliminary Studies with Il-1, Il-2, Il-6, Gm-Csf and Interferon-Gamma.” Cytokine 6 (1): 92-101.

Anderson, P. M., E. Katsanis, A. S. Leonard, D. Schow, C. M. Loeffler, M. B. Goldstein and A. C. Ochoa (1990). “Increased Local Antitumor Effects of Interleukin 2 Liposomes in Mice with Mca-106 Sarcoma Pulmonary Metastases.” Cancer Res 50 (6): 1853-1856.

Cartron, G., L. Zhao-Yang, M. Baudard, T. Kanouni, V. Rouille, P. Quittet, B. Klein and J. F. Rossi (2008). “Granulocyte-Macrophage Colony-Stimulating Factor Potentiates Rituximab in Patients with Relapsed Follicular Lymphoma: Results of a Phase Ii Study.” J Clin Oncol 26 (16): 2725-2731.

Debs, R. J., H. J. Fuchs, R. Philip, E. N. Brunette, N. Duzgunes, J. E. Shellito, D. Liggitt and J. R. Patton (1990). “Immunomodulatory and Toxic Effects of Free and Liposome-Encapsulated Tumor Necrosis Factor Alpha in Rats.” Cancer Res 50 (2): 375-380.

Gruber, M., K. Fleiss, E. Porpaczy, C. Skrabs, A. W. Hauswirth, A. Gaiger, K. Vanura, D. Heintel, M. Shehata, C. Einberger, R. Thalhammer, C. Fonatsch and U. Jager (2011). “Prolonged Progression-Free Survival in Patients with Chronic Lymphocytic Leukemia Receiving Granulocyte Colony-Stimulating Factor During Treatment with Fludarabine, Cyclophosphamide, and Rituximab.” Ann Hematol 90 (10): 1131-1136.

Hernandez-Ilizaliturri, F. J., V. Jupudy, J. Ostberg, E. Oflazoglu, A. Huberman, E. Repasky and M. S. Czuczman (2003). “Neutrophils Contribute to the Biological Antitumor Activity of Rituximab in a Non-Hodgkin's Lymphoma Severe Combined Immunodeficiency Mouse Model.” Clin Cancer Res 9 (16 Pt 1): 5866-5873.

Hernandez-Ilizaliturri, F. J., V. Jupudy, S. Reising, E. A. Repasky and M. S. Czuczman (2005). “Concurrent Administration of Granulocyte Colony-Stimulating Factor or Granulocyte-Monocyte Colony-Stimulating Factor Enhances the Biological Activity of Rituximab in a Severe Combined Immunodeficiency Mouse Lymphoma Model.” Leuk Lymphoma 46 (12): 1775-1784.

Honsik, C. J., G. Jung and R. A. Reisfeld (1986). “Lymphokine-Activated Killer Cells Targeted by Monoclonal Antibodies to the Disialogangliosides Gd2 and Gd3 Specifically Lyse Human Tumor Cells of Neuroectodermal Origin.” Proc Natl Acad Sci USA 83 (20): 7893-7897.

Kedar, E., E. Braun, Y. Rutkowski, N. Emanuel and Y. Barenholz (1994a). “Delivery of Cytokines by Liposomes. Ii. Interleukin-2 Encapsulated in Long-Circulating Sterically Stabilized Liposomes: Immunomodulatory and Anti-Tumor Activity in Mice.” J Immunother Emphasis Tumor Immunol 16 (2): 115-124.

Kedar, E., H. Gur, I. Babai, S. Samira, S. Even-Chen and Y. Barenholz (2000). “Delivery of Cytokines by Liposomes: Hematopoietic and Immunomodulatory Activity of Interleukin-2 Encapsulated in Conventional Liposomes and in Long-Circulating Liposomes.” J Immunother 23 (1): 131-145.

Kedar, E., O. Palgi, G. Golod, I. Babai and Y. Barenholz (1997). “Delivery of Cytokines by Liposomes. Iii. Liposome-Encapsulated Gm-Csf and Tnf-Alpha Show Improved Pharmacokinetics and Biological Activity and Reduced Toxicity in Mice.” J Immunother 20 (3): 180-193.

Kedar, E., Y. Rutkowski, E. Braun, N. Emanuel and Y. Barenholz (1994b). “Delivery of Cytokines by Liposomes. I. Preparation and Characterization of Interleukin-2 Encapsulated in Long-Circulating Sterically Stabilized Liposomes.” J Immunother Emphasis Tumor Immunol 16 (1): 47-59.

Kushner, B. H. and N. K. Cheung (1989). “Gm-Csf Enhances 3f8 Monoclonal Antibody-Dependent Cellular Cytotoxicity against Human Melanoma and Neuroblastoma.” Blood 73 (7): 1936-1941.

Lasic, D. D. (1993). Liposomes from Physics to Applications. Amsterdam, Amsterdam Elsevier Science Publishers BV.

Lopes de Menezes, D. E., K. Denis-Mize, Y. Tang, H. Ye, J. C. Kunich, E. N. Garrett, J. Peng, L. S. Cousens, A. B. Gelb, C. Heise, S. E. Wilson, B. Jallal and S. L. Aukerman (2007). “Recombinant Interleukin-2 Significantly Augments Activity of Rituximab in Human Tumor Xenograft Models of B-Cell Non-Hodgkin Lymphoma.” J Immunother 30 (1): 64-74.

Meyer, J., L. Whitcomb and D. Collins (1994). “Efficient Encapsulation of Proteins within Liposomes for Slow Release in Vivo.” Biochem Biophys Res Commun 199 (2): 433-438.

Munn, D. H. and N. K. Cheung (1987). “Interleukin-2 Enhancement of Monoclonal Antibody-Mediated Cellular Cytotoxicity against Human Melanoma.” Cancer Res 47 (24 Pt 1): 6600-6605.

Niitsu, N., M. Khori, M. Hayama, K. Kajiwara, M. Higashihara and J. Tamaru (2005). “Phase I/Ii Study of the Rituximab-Epoct Regimen in Combination with Granulocyte Colony-Stimulating Factor in Patients with Relapsed or Refractory Follicular Lymphoma Including Evaluation of Its Cardiotoxicity Using B-Type Natriuretic Peptide and Troponin T Levels.” Clin Cancer Res 11 (2 Pt 1): 697-702.

Ottonello, L., A. L. Epstein, P. Dapino, P. Barbera, P. Morone and F. Dallegri (1999). “Monoclonal Lym-1 Antibody-Dependent Cytolysis by Neutrophils Exposed to Granulocyte-Macrophage Colony-Stimulating Factor: Intervention of Fcgammarii (Cd32), Cd11b-Cd18 Integrins, and Cd66b Glycoproteins.” Blood 93 (10): 3505-3511.

Rourke, A. M., Y. Cha and D. Collins (1996). “Stabilization of Granulocyte Colony-Stimulating Factor and Structurally Analogous Growth Factors by Anionic Phospholipids.” Biochemistry 35 (36): 11913-11917.

Stockmeyer, B., D. Elsasser, M. Dechant, R. Repp, M. Gramatzki, M. J. Glennie, J. G. van de Winkel and T. Valerius (2001). “Mechanisms of G-Csf- or Gm-Csf-Stimulated Tumor Cell Killing by Fc Receptor-Directed Bispecific Antibodies.” J Immunol Methods 248 (1-2): 103-111.

van der Kolk, L. E., M. de Haas, A. J. Grillo-Lopez, J. W. Baars and M. H. van Oers (2002). “Analysis of Cd20-Dependent Cellular Cytotoxicity by G-Csf-Stimulated Neutrophils.” Leukemia 16 (4): 693-699.

van der Kolk, L. E., A. J. Grillo-Lopez, J. W. Baars and M. H. van Oers (2003). “Treatment of Relapsed B-Cell Non-Hodgkin's Lymphoma with a Combination of Chimeric Anti-Cd20 Monoclonal Antibodies (Rituximab) and G-Csf: Final Report on Safety and Efficacy.” Leukemia 17 (8): 1658-1664.

WO 2009/075582

WO 2010/143969

WO 2010/143970

We claim:

1. A particulate or vesicular material, comprising
an immunomodulator for use in combination therapy with antibodies in treatment of a condition or a disease, wherein said antibody is not conjugated or directly associated with the particulate or vesicular material.

2. The material of claim 1, further comprising at least one phospholipid, phosphatidylethanolamine (PE), phosphatidylglycerol (PG), phosphatidylserine (PS), or any combination thereof.

3. The material of claim 1, further comprising a phosphatidylethanolamine (PE).

4. The material of claim 2, wherein the phospholipid has an acyl chain comprising at least 16 carbon atoms.

5. The material of claim 2, wherein the phospholipid is unsaturated.

6. The material of claim 2, wherein the phospholipid or PE is 1,2-Dioleoyl-sn-Glycero-3-Phosphoethanolamine (DOPE) and/or 1-stearoyl-2-oleoyl-sn-glycero-3-phosphoethanolamine (SOPE).

7. The material of claim 2, wherein the phospholipid or PE is 1,2-Dioleoyl-sn-Glycero-3-Phosphoethanolamine (DOPE).

8. The material of claim 7, wherein the PE or DOPE concentration is at least 50 mol %.

9. The material of claim 1, further comprising polyethylene glycol (PEG) or a derivate thereof.

10. The material of claim 9, wherein the PEG is 1,2-distearoyl-sn-glycero-3-phosphoethanolamine-N-[methoxy(polyethylene glycol)-2000] (DSPE-PEG2000) or 1,2-distearoyl-sn-glycero-3-phosphoethanolamine-N-[methoxy(polyethylene glycol)-5000] (DSPE-PE-5000).

11. The material of claim 9, wherein the PEG concentration is at least 8 mol %.

12. The material of claim 1, wherein said material has an average diameter within the range 50nm to 1200 nm.

13. The material of claim 1, wherein the material has an average diameter within the range 80-510 nm.

14. The material of claim 1, further comprising a cholesterol.

15. The material of claim 1, wherein said material is a liposome.

16. The material of claim 15, wherein the liposome consists of an immunomodulator and DOPE:PEG:CHOL at molar percentages 62:8:30 or 58:12:30 or 54:16:30.

17. The material of claim 1, wherein the immunomodulator is a cytokine.

18. The material of claim 17, wherein the cytokine is a colony-stimulating factor (CSF), interferon (IFN), interleukin (IL), a tumour necrosis factor (TNF), or any combination thereof.

19. The material of claim 18, wherein the CSF is granulocyte monocyte-colony stimulating factor (GM-CSF), granulocyte-colony stimulating factor (G-CSF), or monocyte-colony stimulating factor (M-CSF).

20. The material of claim 17, wherein the cytokine or IL is IL-2 or IL-4.

21. The material of claim 17, wherein the cytokine or IL is aldesleukin.

22. The material of claim 1, wherein the antibody is an IgG antibody.

23. The material of claim 1, wherein the antibody is a therapeutic monoclonal antibody.

24. The material of claim 1, wherein the antibody targets CD20, CD52, CD3, CD4, CD5, CD8, CD19, CD22, CD38, CD138, HER2, ErbB2, CD11, CD30, CD33, CD52, CD25, vascular endothelial growth factor (VEGF), epidermal growth factor receptor (EGFR), Insulin-like Growth Factor 1 (IGF1) receptor or CTLA-4.

25. The material of claim 1, wherein the antibody is abciximab, adalimumab, alemtuzumab, atlizumab, basiliximab, belimumab, bevacizumab, brentuximab vedotin, canakinumab, cetuximab, certolizumab pegol, cixutumumab, daclizumab, denosumab, eculizumab, efalizumab, gemtuzumab, golimumab, ibritumomab tiuxetan, infliximab, ipilimumab (MDX-101), muromonab-CD3, natalizumab, necitumunab, obinutuzumab (GA-101), ocaratuzumab (AME-133v), ocrelizumab, ofatumumab, omalizumab, palivizumab, panitumumab, pertuzumab, PRO131921, ranibizumab, rituximab, SBI-087, tocilizumab, TRU-015, tositumomab, trastuzumab, veltuzumab, or any combination thereof.

26. The material of claim 1, wherein the condition or disease is cancer, cardiovascular disease, autoimmune disorders, transplant rejection, infectious diseases, inflammatory diseases, degenerative diseases, haematological diseases, myalgic encephalopathy, chronic fatigue syndrome, or post viral fatigue syndrome.

27. The material of claim 1, wherein the condition or disease is cancer.

28. A pharmaceutical composition comprising an antibody and a particulate or vesicular material comprising an immunomodulator, wherein the antibody is not stably conjugated with said material.

29. The pharmaceutical composition of claim 28, wherein the particulate material comprises a particulate or vesicular material comprising said immunomodulator for use in combination therapy with antibodies in treatment of a condition or a disease, wherein said antibody is not conjugated or directly associated with the particulate or vesicular material.

30. A kit comprising an antibody and the material of claim 1.
